(19) United States
(12) Patent Application Publication
(10) Pub. No.: US 2026/0191437 A1
(43) Pub. Date: Jul. 9, 2026

(54) NON-INVASIVE ANALYTE SENSOR FOR ON-SKIN WEARING

(71) Applicant: Roche Diabetes Care GmbH, Mannheim (DE)

(72) Inventor: Kirill Sliozberg, Mannheim (DE)

(21) Appl. No.: 19/558,843

(22) Filed: Mar. 6, 2026

Related U.S. Application Data
(63) Continuation of application No. PCT/EP2024/075164, filed on Sep. 10, 2024.

(30) Foreign Application Priority Data
Sep. 13, 2023 (EP) 23197222.5

(51) Int. Cl. A61B 5/145; A61B 5/00; A61B 5/1455; A61B 5/1477; A61B 5/1486

(52) U.S. Cl. CPC A61B 5/14532; A61B 5/14517; A61B 5/1455; A61B 5/1477; A61B 5/1486; A61B 5/681

(57) ABSTRACT

A non-invasive analyte sensor for on-skin wearing is described. The sensor includes at least one analyte-responsive layer exhibiting at least one physical property depending on a quantity of the analyte, wherein the analyte-responsive layer has a skin contacting surface configured for directly or indirectly contacting the skin; and at least one conversion layer.

### CROSS REFERENCE TO RELATED APPLICATIONS

This is a continuation of application serial no. PCT/EP2024/075164 filed Sep. 10, 2024, which claims priority to EP 23 197 222.5 filed Sep. 13, 2023, the disclosures of both of which are hereby incorporated herein by reference.

### BACKGROUND

### Technical Field

The present application relates to non-invasive analyte sensors.

### Background Art

Non-invasive continuous monitoring systems for detection of an analyte such as for the detection of glucose are generally known. Such systems may be worn on-skin such as may be applied to the skin by using at least one patch or may be worn such as a wristwatch.

For example, non-invasive monitoring systems for detection of an analyte are known which detect the analyte while the analyte is not being actively withdrawn from the detection volume. However, these non-invasive monitoring systems suffer with the problem of slow resorption of glucose by the skin, so that the system would show the accumulated glucose concentration, which does not correspond to the actual glucose levels in, e.g., the interstitial fluid (ISF).

Moreover, equilibrium sensors for glucose monitoring are known. However, an equilibrium sensor does not actively consume glucose and can, thus, only operate under such conditions, where the glucose concentration actively changes, e.g., in-vivo and/or minimal-invasive, or in some reactors. Correspondingly, such kind of arrangement is not suitable for a non-invasive on-skin sensor, which measures glucose levels in the bodily leachates, such as sweat.

U.S. Pat. Nos. 11,395,630 B2 and 11,369,295 B2 describe devices for monitoring an analyte concentration in tear fluid.

U.S. Pat. No. 10,034,625 B1 discloses different embodiments of an analyte sensor to detect an analyte in perspiration or interstitial fluid comprising a substrate with an aptamer configured to obtain one or more measurements related to the analyte.

### SUMMARY

The present disclosure concerns a non-invasive analyte sensor, a non-invasive analyte sensing system, a method for determining a concentration of an analyte in a sample and a computer program. The method and devices according to the present disclosure may be used for detecting at least one analyte present in bodily leachates. In particular, the method and devices are applied in the field of detecting one or more analytes such as glucose or other analytes in bodily leachates, such as sweat, both in the field of professional diagnostics, in the field of hospital point of care, in the field of personal care and in the field of home monitoring. However, other fields of application are feasible.

The present disclosure provides a non-invasive analyte sensor, a non-invasive analyte sensing system, a method for determining a concentration of an analyte in a sample, which at least partially avoid the shortcomings of known devices and methods of this kind and which at least partially address the above-mentioned challenges. Specifically, devices and methods shall be provided which allow for a precise and non-invasive determination of an analyte concentration.

This problem is addressed by non-invasive analyte sensor, a non-invasive analyte sensing system, a method for determining a concentration of an analyte in a sample and a computer program with the features of the independent claims. Advantageous embodiments which might be realized in an isolated fashion or in any arbitrary combinations are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, a non-invasive analyte sensor for on-skin wearing is disclosed.

The non-invasive analyte sensor comprises:
at least one analyte-responsive layer exhibiting at least one physical property depending on a quantity of the analyte, wherein the analyte-responsive layer has a skin contacting surface configured for directly or indirectly contacting the skin;
at least one conversion layer.

The term “analyte” as used herein is a broad term and is to be given its ordinary and customary meaning to a person of ordinary skill in the art and is not to be limited to a special or customized meaning. The term specifically may refer, without limitation, to an arbitrary element, component or compound which may be present in a fluid, specifically in a bodily fluid, and the concentration of which may be of interest for a user. Specifically, the analyte may be or may comprise an arbitrary chemical substance or chemical compound which may take part in the metabolism of the user, such as at least one metabolite. As an example, the at least one analyte may be selected from the group consisting of glucose, lactate, ascorbate and any other analyte, for which an analyte-responsive layer can be designed and which can be followingly converted by a suitable conversion layer. For example, the analyte is glucose. Additionally or alternatively, however, other types of analytes may be determined and/or any combination of analytes may be determined. The term “fluid” as used herein is a broad term and is to be given its ordinary and customary meaning to a person of ordinary skill in the art and is not to be limited to a special or customized meaning. The term specifically may refer, without limitation, to biofluids of interest known to comprise or suspected to comprise the analyte. The fluid may be a bodily fluid, for example, a bodily leachate, such as sweat, tears, or saliva. The bodily fluid generally may be contained in a body tissue. The detection of the at least one analyte in the bodily fluid may preferably be determined on skin. The analyte sensor may be an on skin sensor. The term “on skin sensor” as used herein is a broad term and is to be given its ordinary and customary meaning to a person of ordinary skill in the art and is not to be limited to a special or customized meaning. The term specifically may refer, without limitation, to a sensor which is configured for analyte detection on the living tissue at the skin surface.

The term “sensor” as used herein is a broad term and is to be given its ordinary and customary meaning to a person of ordinary skill in the art and is not to be limited to a special or customized meaning. The term specifically may refer, without limitation, to an arbitrary element or device configured for detecting at least one condition or for measuring at least one measurement variable. The term “analyte sensor” as used herein is a broad term and is to be given its ordinary and customary meaning to a person of ordinary skill in the art and is not to be limited to a special or customized meaning. The term specifically may refer, without limitation, to a sensor configured for detecting quantitatively or qualitatively at least one analyte. The non-invasive analyte sensor may be configured for continuous analyte monitoring.

The term “non-invasive” as used herein is a broad term and is to be given its ordinary and customary meaning to a person of ordinary skill in the art and is not to be limited to a special or customized meaning. The term specifically may refer, without limitation, to an analyte sensor operating outside of the user's body, in particular without penetration and/or penetration of the skin.

The analyte sensor is configured for on-skin wearing. The term “on-skin wearing” as used herein is a broad term and is to be given its ordinary and customary meaning to a person of ordinary skill in the art and is not to be limited to a special or customized meaning. The term specifically may refer, without limitation, to the fact the analyte sensor is mountable and/or attachable to an outer skin surface and/or that the analyte sensor is at least partially in contact with an outer skin surface. The outer skin surface may be the epidermis. For performing an analyte detection, the analyte sensor may be brought in contact with the outer skin surface, e.g., such that the analyte sensor can have direct contact with sweat pores on the epidermis. The analyte sensor may be mountable and/or attachable to an outer skin surface by using at least one patch and/or the analyte sensor may be a part of a patch. The analyte sensor may be skin worn by a user. The analyte sensor may be worn as wristwatch, bracelet, and the like. For example, the analyte sensor may be a patch, firmly contacting the skin surface. The analyte sensor may be an element of a non-invasive analyte sensing system, as will be described in more detail below. The non-invasive analyte sensing system may comprise at least one read-out device, e.g., at least one optical read-out device. For example, the read-out device may be a dedicated firmly attached transmitter for high quality continuous read-outs. For example, the read-out device may be or may be comprised by a smart watch, fitness band for continuous monitoring. The read-out device may be an external device for single readouts such as for flash glucose monitoring. Additionally or alternatively, the read-out may be performed by bare eyes, e.g., for rough estimation. This may be possible if the analyte sensor patch changes its color in the visible range and has sufficient intensity.

The term “user” as used herein is a broad term and is to be given its ordinary and customary meaning to a person of ordinary skill in the art and is not to be limited to a special or customized meaning. The term specifically may refer, without limitation, to a human being or an animal, independent from the fact that the human being or animal, respectively, may be in a healthy condition or may suffer from one or more diseases. As an example, the user may be a human being or an animal suffering from diabetes. However, additionally or alternatively, the disclosure may be applied to other types of users.

The term “analyte-responsive layer” as used herein is a broad term and is to be given its ordinary and customary meaning to a person of ordinary skill in the art and is not to be limited to a special or customized meaning. The term specifically may refer, without limitation, to a layer exhibiting at least one physical property depending on a quantity of the analyte. For example, the analyte-responsive layer may be configured for changing at least one physical property, e.g., an optical property, e.g., depending on a quantity of the analyte. For example, the analyte may be glucose and the analyte-responsive layer may be a glucose-responsive layer.

The analyte-responsive layer may be an equilibrium analyte-responsive layer. The term “equilibrium analyte-responsive layer” as used herein is a broad term and is to be given its ordinary and customary meaning to a person of ordinary skill in the art and is not to be limited to a special or customized meaning. The term specifically may refer, without limitation, to an analyte-responsive layer comprising an equilibrium within the used detection chemistry. In the analyte-responsive layer, an equilibrium exists between uncharged and charged forms of analyte binding molecules and counter ions. In case of addition of analyte, the charged form binds it, leaving the counter ion. The equilibrium between uncharged and the charged forms will be continuously re-established. The amount of the free counter ions, thus, is proportional to the analyte quantity. The concentration of the free counter ions affects the physico-chemical property of the analyte-responsive layer. For example, the equilibrium may related to two forms of the boronic acid-based analyte-responsive layer. However, embodiments are thinkable in which the analyte-responsive layer is not an equilibrium analyte-responsive layer but works in a different way.

The non-invasive analyte sensor may be an equilibrium sensor. Thus, it does not actively consume the analyte, e.g., glucose, and can only work under conditions, where the bulk glucose concentration actively changes, e.g., in-vivo. Correspondingly, usually such equilibrium sensor cannot be used for a non-invasive on-skin sensor, which measures glucose levels in the bodily leachates, such as sweat. The reason is slow resorption of glucose by the skin, so that the sensor would show the accumulated glucose concentration, which does not correspond to the actual glucose levels in, e.g., ISF. The present disclosure allows for providing a set-up, where the glucose concentration within the detection volume corresponds to the bulk glucose concentration. The term “equilibrium sensor” as used herein is a broad term and is to be given its ordinary and customary meaning to a person of ordinary skill in the art and is not to be limited to a special or customized meaning. The term specifically may refer, without limitation, to an analyte sensor having an axial equilibrium. In particular, the axial equilibrium relates to the analyte concentration between the bulk concentration, coming with the sample, e.g., leachate, and zero (within tolerances) as the whole analyte is being consumed by the conversion layer. Thus, an axial axis from proximal to distal, the concentration of the analyte drops from the maximum, as delivered by the sample and zero, where it is consumed by the conversion layer. The analyte-responsive layer may be placed somewhere on said axis and it measures a signal proportional to the input at the proximal end. For example, the analyte sensor may comprise at its proximal end the analyte-responsive layer and more distal from the skin the conversion layer.

For example, the analyte-responsive layer comprises at least one analyte-responsive hydrogel layer. The term “hydrogel” as used herein is a broad term and is to be given its ordinary and customary meaning to a person of ordinary skill in the art and is not to be limited to a special or customized meaning. The term specifically may refer, without limitation, to a solid material comprising a three-dimensional polymeric matrix or network in the medium of a liquid.

The non-invasive analyte sensor may operate on a detection principle which uses based on a glucose-responsive hydrogel (GRH) bearing a phenylborate derivatives. However, embodiments of the non-invasive analyte sensor are not limited by using of the phenylborate derivate based GRHs and thus is not limited to diol detection only. Phenylboronic acid and its derivatives are known to form reversible covalent complexes with diol units, such as glucose. GRH reversibly bind diols and change its at least one optical property, depending on further functional groups comprising the GRH.

For example, the GRH comprises boronic acid. For example, the GRH may change its fluorescence properties upon diol binding. The change of the fluorescence may be measured by illuminating the GRH with light, e.g., excitation light having at least certain excitation wavelength spectrum, and measuring the fluorescence as a response to the illumination, e.g., the duration and/or intensity thereof. The excitation wavelength spectrum may depend on the chemical composition of the GRH. The excitation wavelength spectrum may lie in the visible range, e.g., as described in 10.1023/b:jofl.0000039338.16715.48, and/or in the NIR, e.g., as described in https://doi.org/10.1021/nn204323f.

For example, the GRH comprises boronic acid. For example, the GRH comprises boronic acid azobenzene. For example, the analyte-responsive hydrogel layer comprises boronic acid-substituted azobenzene derivatives. For example, the analyte-responsive hydrogel layer comprises o-boronic acid-substituted azobenzene. O-boronic acid-substituted azobenzenes are known to drastically change its absorption spectra in visible range upon diol binding, as described in Egawa et al. “Colorimetric Sugar Sensing Using Boronic Acid-Substituted Azobenzenes”, Materials 2014, 7(2), 1201-1220; https://doi.org/10.3390/ma7021201.

The analyte-responsive layer has a skin contacting surface configured for directly or indirectly contacting the skin. The term “skin contacting surface” as used herein is a broad term and is to be given its ordinary and customary meaning to a person of ordinary skill in the art and is not to be limited to a special or customized meaning. The term specifically may refer, without limitation, to a surface of the analyte-responsive layer configured for contacting with the skin of the user, either directly or indirectly. For example, the skin contacting surface may be the skin-contacting layer directly in contact with the skin of the user. Alternatively, additional layers between the skin contacting surface and the skin may be used, e.g., the skin contacting surface may be contacted indirectly via at least one additional element such as at least one additional functional layer, i.e., buffering hydrogel layer or light reflective layer or filtering layer.

The term “conversion layer” as used herein is a broad term and is to be given its ordinary and customary meaning to a person of ordinary skill in the art and is not to be limited to a special or customized meaning. The term specifically may refer, without limitation, to a layer configured for chemically or electrochemically converting the analyte. The conversion layer may actively remove the analyte from the analyte-responsive layer, thereby continuously re-creating an equilibrium analyte concentration. The analyte concentration may lie somewhere on the axis between the continuously varying analyte concentration on the leachate and “zero”, where the analyte is completely converted. This equilibrium in combination with the integrated conversion layer and electron acceptor, as will be outlined below, can allow an optical measurement. The analyte-responsive layer may be used for analyte detection, but the conversion layer may be required as an additional mechanism, which enable continuous equilibrium re-establishment.

The conversion layer may be designed as a single stack of at least one or a plurality of functional layers. However, other embodiments are thinkable. For example, the conversion layer may comprise two or more laterally distributed functional layers.

The non-invasive analyte sensor may comprise at least one electron acceptor. The conversion layer may comprise an electron acceptor. The electron acceptor may be integrated into the conversion layer. The electron acceptor may be a constituent part of the conversion layer. This can allow optical measurement. Depending on its design, the electron acceptor may be made a separate layer. The term “electron acceptor” as used herein is a broad term and is to be given its ordinary and customary meaning to a person of ordinary skill in the art and is not to be limited to a special or customized meaning. The term specifically may refer, without limitation, to any redox active chemical compound, having sufficiently positive redox potential, to accept electrons generated by the conversion layer during analyte, e.g., glucose, conversion. The electron acceptor can be a constituent part of the analyte sensor, e.g., of the conversion layer or an additional layer such as on top of the conversion layer. For example, the electron acceptor is a Ag/AgCl layer. Additionally or alternatively, the electron acceptor may be an externally supplied commodity such as molecular oxygen.

The conversion layer may be enzyme-comprising or be non-enzymatic. For example, the conversion layer may comprise enzyme molecules for converting of the analyte. The conversion layer may comprise glucose oxidase (GOx) or glucose dehydroginase (GDH). In the case of the GOx, molecular oxygen from the ambient air can be used as natural electron acceptor. In this case, all layers above the conversion layer may be designed to be at least partially oxygen permeable to supply oxygen to GOx. The reaction product, hydrogen peroxide, may, preferably, be directly decomposed in order to avoid its oxidative action towards enzyme. The conversion layer may comprise some catalyst material, such as manganese oxide or platinum nanoparticles. In the case of GDH, which is an oxygen independent enzyme, another electron acceptor is desired, for instance silver chloride. In order to transfer electrons from the enzyme (co-factor) another mediator may be used. Therefore, the enzyme may be chemically wired by at least one redox hydrogel e.g. at least one Os-complex modified hydrogel.

The conversion layer may be non-enzymatic. For example, the conversion layer comprises at least one material selected from the material classes of metal-organic frameworks, nanoparticle metal salts of low solubility, like tin-nickel sulfide, graphene oxide, carbon nanotubes and other.

The analyte sensor may comprise at least one light reflective layer. The light reflective layer may be permeable for the analyte. The term “light reflective layer” as used herein is a broad term and is to be given its ordinary and customary meaning to a person of ordinary skill in the art and is not to be limited to a special or customized meaning. The term specifically may refer, without limitation, to at least one layer configured for at least partially redirecting impinging light. For example, the light reflective layer may be or may comprise at least one reflective membrane. The light reflective layer may be arranged underneath of the analyte-responsive layer.

The term “light” as used herein is a broad term and is to be given its ordinary and customary meaning to a person of ordinary skill in the art and is not to be limited to a special or customized meaning. The term specifically may refer, without limitation, to electromagnetic radiation, e.g., in one or more of the infrared or the visible spectral range. The term “visible spectral range”, generally may refer to a spectral range of 380 nm to 760 nm. The term “infrared spectral range” (IR) generally may refer to electromagnetic radiation of 760 nm to 1000 μm, wherein the range of 760 nm to 1.5 μm is usually denominated as “near infrared spectral range” (NIR) while the range from 1.5 μm to 15 μm is denoted as “mid infrared spectral range” (MidIR) and the range from 15 μm to 1000 μm as “far infrared spectral range” (FIR). For example, light used for the typical purposes of the present disclosure is light in the visible range or the IR spectral range, e.g., the NIR range. A vast number of boronic acid based GRH composition may be possible, covering all possible wavelength ranges.

For example, the analyte sensor comprises the following layers in the named order, starting most proximal from the skin to further distal layers. The analyte sensor may comprise as most proximal layer to the skin the analyte-responsive layer, e.g., designed as an optical GRH layer. On top of the analyte-responsive layer the conversion layer, e.g., a wired enzyme layer, may be arranged. On top of the conversion layer, the electron acceptor, e.g., an Ag/AgCl layer, may be used. For example, the analyte sensor may be manufactured by providing a continuous film or membrane film, which may be the top layer (distal) of the resulting patch. The continuous film or membrane film may then be coated with corresponding chemicals, e.g., by using one or more of the following techniques, such as slot-die coating, screen printing, off-set printing, but also something like PVD or CVD, e.g., for modifying of the film with ITO, PEDOT, or FTO as conductive layers or, e.g., aluminum film for reflectance.

For example, the analyte sensor comprises the following layers in the named order, starting most proximal from the skin to further distal layers. The analyte sensor may comprise as most proximal layer to the skin the light reflective layer. On top of the light reflective layer, the analyte-responsive layer may be arranged. On top of the analyte-responsive layer the conversion layer, e.g. a wired enzyme layer, may be arranged. On top of the conversion layer, the electron acceptor, e.g., an Ag/AgCl layer, may be used. Alternatively, e.g., for GRH working in visible spectral range, the Ag/AgCl layer may not lie in the pathway of an optical measurement system, as it will absorb the visible light. The sensor may comprise a transparent conductive layer, e.g., PEDOT or ITO/FTO coated transparent plastics or foils. The electron acceptor, e.g., the Ag/AgCl layer, may be arranged laterally shifted with respect to the analyte-responsive layer and the light pathway.

The analyte sensor proposed herein can allow improving a non-invasive equilibrium sensor for continuous glucose monitoring. The analyte sensor can allow for providing an optically measurable property of the analyte-responsive layer using a simple setup, e.g., without a plurality of electrodes and/or power supply. To re-establish an equilibrium within the analyte-responsive layer, said layer is combined with a conversion layer as well as an electron acceptor. Thus, this setup comprises a single electrode while ensuring re-establishment of an equilibrium state of the analyte-responsive layer. Such a simple measuring setup suitable for on-skin use significantly improves non-invasive equilibrium sensors for continuous glucose monitoring.

The analyte sensor as standalone device can be used for determining an analyte concentration, e.g., an estimation thereof. For example, a color change and/or color intensity can be determined by bare eyes and can give at least an estimation of the analyte concentration.

In a further aspect, a non-invasive analyte sensing system, e.g., for on-skin wearing is disclosed. The system comprises:
at least one non-invasive analyte sensor according to the present disclosure,
at least one optical measurement system configured for detecting the physical property of the analyte-responsive layer;
at least one processing unit configured for determining an analyte concentration by evaluating the physical property.

With respect to definitions and embodiments of the non-invasive analyte sensor reference is made to the description of the non-invasive analyte sensor described in a first aspect or as described in more detail below.

As further used herein, the term “system” refers to an arbitrary set of interacting or interdependent component parts forming a whole. Specifically, the components may interact with each other in order to fulfill at least one common function. The at least two components may be handled independently or may be coupled or connectable. Thus, the term “sensing system” generally refers to a group of at least two elements or components which are capable of interacting in order to perform at least one analytical detection, specifically at least one analytical detection of at least one analyte of a sample. The sensing system may be an apparatus, specifically comprising at least two components. The sensing may comprise a process of continuously acquiring data and deriving desired information therefrom without user interaction. For this purpose, a plurality of measurement signals are generated and evaluated, wherefrom the desired information is determined. Herein, the plurality of measurement signals may be recorded within fixed or variable time intervals or, alternatively or in addition, at an occurrence of at least one prespecified event. For example, the analyte sensor as used herein may, especially, be configured for a continuous monitoring of one or more analytes, such as for managing, monitoring, and controlling a diabetes state.

The term “optical measurement system” as used herein is a broad term and is to be given its ordinary and customary meaning to a person of ordinary skill in the art and is not to be limited to a special or customized meaning. The term specifically may refer, without limitation, to at least one optical detector. The optical measurement system may be configured for one or more of detecting, measuring and monitoring, of at least one parameter, qualitatively and/or quantitatively, relating to the physical property, e.g., the optical property, of the analyte-responsive layer.

The optical measurement system may comprise at least one light source. The term “light source” as used herein is a broad term and is to be given its ordinary and customary meaning to a person of ordinary skill in the art and is not to be limited to a special or customized meaning. The term specifically may refer, without limitation, to an arbitrary device configured for generating or providing light in the sense of the above-mentioned definition. The light source specifically may be or may comprise at least one light-emitting diode (LED). The optical measurement system may comprise at least one optical filter. Alternatively to an artificial light source, ambient light may be used. The light source may be configured for illuminating the analyte sensor. The term “illuminate”, as used herein, is a broad term and is to be given its ordinary and customary meaning to a person of ordinary skill in the art and is not to be limited to a special or customized meaning. The term specifically may refer, without limitation, to the process of exposing at least one element to light. The illumination may comprise an excitation.

The term “physical property of the analyte-responsive layer” as used herein is a broad term and is to be given its ordinary and customary meaning to a person of ordinary skill in the art and is not to be limited to a special or customized meaning. The term specifically may refer, without limitation, to a physically measurable property such as an optically measurable property which is determined as a function of wavelengths such as one or more of at least one property characterizing at least one reflectance property, at least one transmission property or at least one absorption property. For example, the physical property may change depending on the amount of the analyte. For example, the optical property may be an intensity, e.g., a fluorescence intensity. For example, the optical property may be a fluorescence duration. The optical property may be determined for one or more wavelengths. As will be outlined in more detail below, the system may comprise a spectrophotometer device. The spectrophotometer device may be configured for recording a signal intensity with respect to the corresponding wavelength of a spectrum or a partition thereof, such as a wavelength interval, wherein the signal intensity may, specifically, be provided as an electrical signal which may be used for further evaluation.

For example, the optical property may be measurable by recording at least one absorption spectrum of the analyte-responsive layer. The term “absorption spectrum” as used herein is a broad term and is to be given its ordinary and customary meaning to a person of ordinary skill in the art and is not to be limited to a special or customized meaning. The term specifically may refer, without limitation, to a measure of the absorption of light as a function of frequency or wavelength due to its interaction with a sample, in this case the analyte response layer. The absorption spectrum of the analyte response layer may be the fraction of incident light absorbed by the analyte response layer over a range of wavelengths of light. The optical measurement system may comprise at least one spectrophotometer device. The term “spectrophotometer device” as used herein is a broad term and is to be given its ordinary and customary meaning to a person of ordinary skill in the art and is not to be limited to a special or customized meaning. The term specifically may refer, without limitation, to an optical device configured for acquiring at least one item of spectral information. The spectrophotometer device may be an absorption spectrometer.

For example, the analyte-responsive layer may be designed as an optical GRH layer changing its fluorescence duration upon diol binding. The optical measurement system may illuminate the GRH layer by an excitation wavelength spectrum. The optical measurement system may measure an intensity and/or a duration of the fluorescence.

The optical measurement system may comprise at least one camera. The term “camera” as used herein is a broad term and is to be given its ordinary and customary meaning to a person of ordinary skill in the art and is not to be limited to a special or customized meaning. The term specifically may refer, without limitation, to a device having at least one imaging element configured for recording or capturing spatially resolved one-dimensional, a two-dimensional or even three-dimensional optical information. As an example, the camera may comprise at least one camera chip, such as at least one CCD chip and/or at least one CMOS chip. For example, the camera may be a color camera comprising at least three color pixels. The camera may be a color CMOS camera. For example, the camera may be a black and white CMOS. The camera may comprise at least one color camera and at least one black and white camera, such as a black and white CMOS. The camera may comprise at least one black and white CMOS chip. The camera generally may comprise a one-dimensional or two-dimensional array of image sensors, such as pixels. As an example, the camera may comprise at least 10 pixels in at least one dimension, such as at least 10 pixels in each dimension. It shall be noted, however, that other cameras are also feasible. For example, the camera may be or may comprise an IR sensitive sensor.

The camera may be a camera of a mobile communications device. For example, the disclosure can be used in mobile applications such as notebook computers, tablets or, specifically, cell phones such as smart phones. Thus, specifically, the camera may be part of a mobile device which, besides the at least one camera, comprises one or more data processing units such as one or more data processors. Other cameras, however, are feasible.

The camera, besides at least one camera chip or imaging chip, may comprise further elements, such as one or more optical elements, e.g., one or more lenses. As an example, the camera may be a fix-focus camera, having at least one lens which is fixedly adjusted with respect to the camera. Alternatively, however, the camera may also comprise one or more variable lenses which may be adjusted, automatically or manually.

The camera may be configured for generating at least one signal, such as an analogue and/or a digital signal, the signal providing information on the at least one parameter measured by the camera. The signal may directly or indirectly be provided by the camera to the processing unit, such that the camera and the processing unit may be directly or indirectly connected.

The camera specifically may be a color camera. Thus, such as for each pixel, color information may be provided or generated, such as color values for three colors R, G, B. A larger number of color values is also feasible, such as four colors for each pixel, for example R, G, G, B. Color cameras are generally known to the skilled person. Thus, as an example, each pixel of the camera chip may have three or more different color sensors, such as color recording pixels like one pixel for red (R), one pixel for green (G) and one pixel for blue (B). For each of the pixels, such as for R, G, B, values may be recorded by the pixels, such as digital values in the range of 0 to 255, depending on the intensity of the respective color. Instead of using color triples such as R, G, B, as an example, quadruples may be used, such as R, G, G, B or C, M, Y, K or the like. The color sensitivities of the pixels may be generated by color filters or by appropriate intrinsic sensitivities of the sensor elements used in the camera pixels. These techniques are generally known to the skilled person.

The optical measurement system may comprise at least one wavelength-selective element, such as at least one of a grating, a prism and a filter, e.g., a length variable filter having varying transmission properties over its lateral extension. The wavelength-selective element may be used for separating incident light into a spectrum of constituent wavelength signals whose respective intensities are determined by employing the camera.

The term “processing unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. The processing unit may be configured for processing basic instructions that drive the computer or system. As an example, the processing unit may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. The processing unit may be a multi-core processor. The processing unit may be or may comprise a central processing unit (CPU). Additionally or alternatively, the processing device may be or may comprise a microprocessor, thus specifically the processor's elements may be contained in one single integrated circuitry (IC) chip. Additionally or alternatively, the processing unit may be or may comprise one or more application-specific integrated circuits (ASICs) and/or one or more field-programmable gate arrays (FPGAs) and/or one or more tensor processing unit (TPU) and/or one or more chips, such as a dedicated machine learning optimized chip, or the like. The processing unit may be configured, such as by software programming, for performing one or more evaluation operations. The processing device may be configured for performing the named step(s). Thus, as an example, the processing unit may comprise a software code stored thereon comprising a number of computer instructions. The processing unit may provide one or more hardware elements for performing one or more of the indicated operations and/or may provide one or more processors with software running thereon for performing one or more of steps.

The processing unit may be configured for determining the physical property, e.g., the optical property such as a change of the optical property, of the analyte-responsive layer. The determining may comprise comparing the determined absorption spectrum to at least one reference spectrum. For example, the reference spectrum may be an absorption spectrum measured during manufacturing of the analyte sensor and/or at the begin of operation, e.g., right after mounting, of the analyte sensor on the skin. The reference spectrum may be stored in at least one database of the analyte sensing system and/or may be obtained from an external database such as from a cloud. For example, the reference spectrum may be a spectrum obtained by at least one further spectrometer, e.g., a reference spectrometer. The analyte sensing system may be calibrated using at least one further device, e.g., a blood glucose meter or another device for continuous glucose monitoring. In this case a spectrum obtained by the analyte sensing system may be correlated with a current analyte level measured by using the further device and make it thus to a reference.

The physical property may be dependent on the analyte concentration, e.g., may be proportional to the analyte concentration. For example, as outlined above, the GRH may change its fluorescence properties upon diol binding. The change of the fluorescence properties may be measured by illuminating the GRH with light, e.g., excitation light having at least certain excitation wavelength spectrum, and measuring the fluorescence as a response to the illumination. One or more properties of the fluorescence may be measured, e.g., a change of the intensity, change of the duration of the fluorescence, intensity decay over time and the like. Such properties may change as a function of the analyte concentration, e.g., as described in https://doi.org/10.1023/B:JOFL.0000039338.16715.48 FIG. 1.

Additionally or alternatively, at least one colorimetric measurement may be performed, e.g., a change in the absorption spectrum may be used. For example, in case of the, e.g., boronic Acid-Substituted Azobenzenes, a colorimetric measurement may be performed, i.e., a change in the absorption spectrum may be monitored. For example, some wavelength range of the absorption spectrum may be used or one or few single wavelengths of the absorption spectrum, e.g., absorption at 521 nm and/or 398 nm for a given compound.

The determined physical property may be evaluated into a concentration value by the processing unit, e.g., by using at least one predetermined relationship between the analyte concentration and the physical property.

The term “to evaluate”, as used herein, is a broad term and is to be given its ordinary and customary meaning to a person of ordinary skill in the art and is not to be limited to a special or customized meaning. The term specifically may refer, without limitation, to the process of processing at least one first item of information in order to generate at least one second item of information thereby.

The non-invasive analyte sensing system may comprise at least one reference field. The reference field may allow for continuous self-calibration of the optical system. The term “reference field” as used herein is a broad term and is to be given its ordinary and customary meaning to a person of ordinary skill in the art and is not to be limited to a special or customized meaning. The term specifically may refer, without limitation, to an arbitrary two-dimensional area which has predetermined optical properties, e.g., a predetermined absorption spectrum. The sensor patch may have one or more additional color reference fields or a separate “color card” may be used. The color card may comprise one or more color reference fields and, optionally, one or more gray color reference fields. The color reference fields may be or may comprise uniformly colored fields having known or predetermined color properties, such as at least one known or predetermined color coordinate in at least one color space. The color card may specifically comprise a plurality of color reference fields, each of the color reference fields having different known or predetermined color properties, specifically different color coordinates in at least one color space. The gray color reference fields may be or may comprise uniformly gray colored fields having known or predetermined gray color properties, such as a known or predetermined shade of gray in between black and white. The color card may specifically comprise a plurality of gray color reference fields, each of the gray color reference fields having different known or predetermined gray color properties, specifically different shades of gray in between black and white. The color reference fields and, optionally, the gray color reference fields, may have an arbitrary two-dimensional shape, such as a rectangle, a square, a polygon, a circle and/or an ellipse. The color card may comprise a substrate, such as a flat substrate, having disposed thereon or therein the one or more color reference fields and, optionally, the one or more gray color reference fields.

The non-invasive analyte sensing system may further comprise at least one temperature sensor. The temperature sensor may determine temperature values, e.g., at predefined times. The temperature sensor may be built in the analyte sensor. The temperature values may allow correcting measurements results, e.g., when measuring fluorescence, as such a measurement is highly dependent on temperature. The temperature sensor may be advantageous for knowing the temperature to adjust for reaction kinetics.

The non-invasive analyte sensing system may comprise a plurality of analytical spots, e.g., laterally distributed at the patch, each comprising an analyte sensor according to the present disclosure. Each of the analyte sensors may be responsive for a different analyte, thereby allowing detection of multiple analytes.

In a further aspect of the present disclosure, a method for determining a concentration of an analyte in a sample using a non-invasive analyte sensing system according to the present disclosure, such as described in one or more of the embodiments enclosed herein, is disclosed. With respect to definitions and embodiments of the non-invasive analyte sensing system reference is made to the description of the non-invasive analyte sensor and the non-invasive analyte sensing system described in a further aspect or as described in more detail below. The method comprises the method steps as given in the corresponding description and as listed as follows. The method steps may be performed in the given order. Further, one or more of the method steps may be performed in parallel and/or in a time overlapping fashion. Further, one or more of the method steps may be performed repeatedly. Further, additional method steps may be present which are not listed.

The method comprising the steps of:
i. contacting non-invasive analyte sensor with a skin surface;
ii. determining the physical property of the analyte-responsive layer;
iii. determining the analyte concentration by evaluating the physical property.

The term “contacting” as used herein is a broad term and is to be given its ordinary and customary meaning to a person of ordinary skill in the art and is not to be limited to a special or customized meaning. The term specifically may refer, without limitation, to a process of exposing the analyte sensor to the fluid, e.g., via attaching and/or mounting the detection electrode to the skin.

The determining of the physical property of the analyte-responsive layer may comprise
illuminating the analyte sensor, e.g. by using light of a predefined wavelength range;
determining at least one absorption spectrum;
determining the physical property by comparing the determined absorption spectrum to at least one reference spectrum.

Further disclosed and proposed herein is a computer program including computer-executable instructions for performing the method according to the present disclosure in one or more of the embodiments enclosed herein when the instructions are executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps i) to iii) as indicated above may be performed by using a computer or a computer network, preferably by using a computer program. In particular, the computer program may execute and/or trigger executing the method steps ii, iii.

Further disclosed and proposed herein is a computer program product having program code means, in order to perform the method according to the present disclosure in one or more of the embodiments described herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a non-transient computer-readable medium including instructions that, when executed by one or more processors, cause the one or more processors to perform the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of the disclosure, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

Summarizing and without excluding further possible embodiments, the following embodiments may be envisaged:

Embodiment 1. A non-invasive analyte sensor for on-skin wearing comprising
at least one analyte-responsive layer exhibiting at least one physical property depending on a quantity of the analyte, wherein the analyte-responsive layer has a skin contacting surface configured for directly or indirectly contacting the skin;
at least one conversion layer; and
optionally, at least one electron acceptor.

Embodiment 2. The non-invasive analyte sensor according to the preceding embodiment, wherein the analyte responsive layer is an equilibrium analyte-responsive layer, wherein the analyte-responsive layer comprises at least one analyte-responsive hydrogel layer, wherein the analyte-responsive hydrogel layer comprises boronic acid, wherein the analyte-responsive hydrogel layer comprises a phenylborate derivative.

Embodiment 3. The non-invasive analyte sensor according to any one of the preceding embodiments, wherein the conversion layer is enzyme-comprising or non-enzymatic.

Embodiment 4. The non-invasive analyte sensor according to any one of the preceding embodiments, wherein the conversion layer comprises enzyme molecules for converting of the analyte, wherein the conversion layer comprises glucose oxidase (GOx) or glucose dehydroginase (GDH).

Embodiment 5. The non-invasive analyte sensor according to any one of the two preceding embodiments, wherein the enzyme is chemically wired by at least one redox hydrogel.

Embodiment 6. The non-invasive analyte sensor according to any one of the preceding embodiments, wherein the electron acceptor is a redox active chemical compound configured for accepting electrons generated by the conversion layer.

Embodiment 7. The non-invasive analyte sensor according to any one of the preceding embodiments, wherein the electron acceptor is comprised by the conversion layer.

Embodiment 8. The non-invasive analyte sensor according to any one of the preceding embodiments, wherein the electron acceptor is an Ag/AgCl layer.

Embodiment 9. The non-invasive analyte sensor according to any one of the preceding embodiments, wherein the analyte sensor comprises at least one light reflective layer, wherein the light reflective layer is permeable for the analyte.

Embodiment 10. The non-invasive analyte sensor according to any one of the preceding embodiments, wherein the analyte is glucose.

Embodiment 11. The non-invasive analyte sensor according to any one of the preceding embodiments, wherein the sample is a bodily leachate such as sweat.

Embodiment 12. A non-invasive analyte sensing system comprising:
at least one non-invasive analyte sensor according to any one of the preceding embodiments,
at least one optical measurement system configured for detecting the physical property of the analyte-responsive layer;
at least one processing unit configured for determining an analyte concentration by evaluating the physical property.

Embodiment 13. The non-invasive analyte sensing system according to the preceding embodiment, further comprising at least one temperature sensor.

Embodiment 14. A method for determining a concentration of an analyte in a sample using a non-invasive analyte sensing system according to any one of the preceding embodiments referring to a non-invasive analyte sensing system, the method comprising the steps of:
i. contacting non-invasive analyte sensor with a skin surface;
ii. determining the physical property of the analyte-responsive layer;
iii. determining the analyte concentration by evaluating the physical property.

Embodiment 15. A computer program comprising program means for performing the method according to the preceding embodiment while the computer program is being executed on a computer or on a computer network.

Embodiment 16. A computer-readable storage medium comprising instructions which, when the instructions are executed by the non-invasive analyte sensing system according to any one of the preceding embodiments referring to a non-invasive analyte sensing system, cause the non-invasive analyte sensing system to perform the method according to embodiment 14.

Embodiment 17. A non-transient computer-readable medium including instructions that, when executed by one or more processors, cause the one or more processors to perform the method according to embodiment 14.

### BRIEF DESCRIPTION OF THE DRAWINGS

Further optional features and embodiments will be disclosed in more detail in the subsequent description of embodiments. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1A and 1B show embodiments of a non-invasive analyte sensing system comprising non-invasive analyte sensor; and

FIG. 2 shows an embodiment of a method for determining a concentration of an analyte in a sample.

Although the exemplifications set out herein illustrate embodiments of the invention in several forms, the embodiments disclosed herein are not intended to be exhaustive or to be construed as limiting the scope of the invention to the precise forms disclosed.

### DETAILED DESCRIPTION

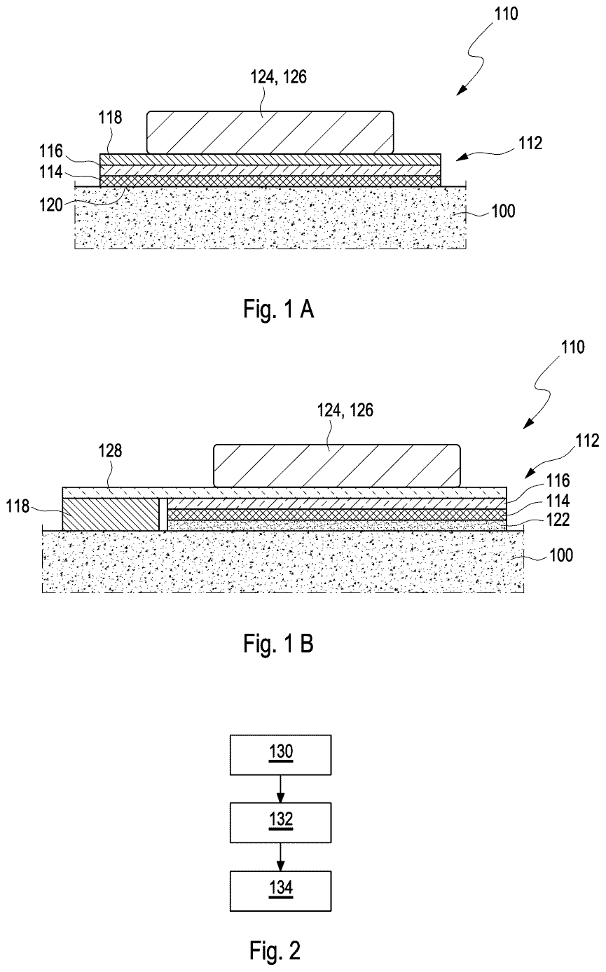

FIGS. 1A and 1B show, in a highly schematic fashion and in a cross sectional view, exemplary embodiments of a non-invasive analyte sensing system 110 comprising a non-invasive analyte sensor 112.

The analyte sensor 112 is configured for on-skin wearing. Skin is denoted with reference number 100 in the FIGS. 1A and 1B. The analyte sensor is 112 mountable and/or attachable to an outer skin surface and/or that the analyte sensor 112 is at least partially in contact with an outer skin surface. The outer skin surface may be the epidermis. For performing an analyte detection, the analyte sensor 112 may be brought in contact with the outer skin surface, e.g., such that the analyte sensor 112 can have direct contact with sweat pores on the epidermis. The analyte sensor 112 may be mountable and/or attachable to an outer skin surface by using at least one patch and/or the analyte sensor 112 may be a part of a patch. The analyte sensor 112 may be skin worn by a user. The analyte sensor 112 may be worn as a wristwatch, bracelet and the like. For example, the analyte sensor 112 may be a patch, firmly contacting the skin surface. The analyte sensor 112 may be an element of the non-invasive analyte sensing system 110. The non-invasive analyte sensing system 110 may comprise at least one read-out device, e.g., at least one optical read-out device. For example, the read-out device may be a dedicated firmly attached transmitter for high quality continuous read-outs. For example, the read-out device may be or may be comprised by a smart watch, fitness band for continuous monitoring. The read-out device may be an external device for single readouts such as for flash glucose monitoring. Additionally or alternatively, the read-out may be performed by bare eyes, e.g., for rough estimation. This may be possible if the analyte sensor patch changes its color in the visible range and has sufficient intensity.

The non-invasive analyte sensor 112 comprises
at least one analyte-responsive layer 114 exhibiting at least one physical property depending on a quantity of the analyte, wherein the analyte-responsive layer 114 has a skin contacting surface configured for directly or indirectly contacting the skin;
at least one conversion layer 116; and
optionally at least one electron acceptor 118.

The analyte-responsive layer 114 may exhibit at least one physical, e.g., optical, property depending on the quantity of the analyte. For example, the analyte may be glucose and the analyte-responsive layer 114 may be a glucose-responsive layer. The analyte-responsive layer 114 may be an equilibrium analyte-responsive layer. The analyte-responsive layer 114 comprises an equilibrium within the used detection chemistry. In the analyte-responsive layer 114, an equilibrium exists between uncharged and charged forms of analyte binding molecules and counter ions. In case of addition of analyte, the charged form binds it, leaving the counter ion. The equilibrium between uncharged and the charged forms will be continuously re-established. The amount of the free counter ions, thus, is proportional to the analyte quantity. The concentration of the free counter ions affects the physico-chemical property of the analyte-responsive layer. However, embodiments are thinkable in which the analyte-responsive layer 114 is not an equilibrium analyte-responsive layer but works in a different way.

The non-invasive analyte sensor 112 may be an equilibrium sensor. Thus, it does not actively consume the analyte, e.g., glucose, and can only work under conditions, where the bulk glucose concentration actively changes, e.g., in-vivo. Correspondingly, usually such equilibrium sensor cannot be used for a non-invasive on-skin sensor, which measures glucose levels in the bodily leachates, such as sweat. The reason is slow resorption of glucose by the skin, so that the sensor would show the accumulated glucose concentration, which does not correspond to the actual glucose levels in, e.g., ISF. The present disclosure allows for providing a set-up, where the glucose concentration within the detection volume corresponds to the bulk glucose concentration. The non-invasive analyte sensor 112 has an axial equilibrium. In particular, the axial equilibrium relates to the analyte concentration between the bulk concentration, coming with the sample, e.g., leachate, and zero (within tolerances) as the whole analyte is being consumed by the conversion layer. Thus, an axial axis from proximal to distal, the concentration of the analyte drops from the maximum, as delivered by the sample and zero, where it is consumed by the conversion layer 116. The analyte-responsive layer 114 may be placed somewhere on said axis and it measures a signal proportional to the input at the proximal end.

For example, the analyte-responsive layer 114 comprises at least one analyte-responsive hydrogel layer. The non-invasive analyte sensor 112 may operate on a detection principle which is based on a glucose-responsive hydrogel (GRH) bearing a phenylborate derivatives. However, embodiments of the non-invasive analyte sensor 112 are not limited by use of the phenylborate derivate based GRHs and thus is not limited to diol detection only. Phenylboronic acid and its derivatives are known to form reversible covalent complexes with diol units, such as glucose. GRH reversibly bind diols and changes its at least one optical property, depending on further functional groups comprising the GRH.

For example, the GRH comprises boronic acid. For example, the GRH may change its fluorescence properties upon diol binding. The change of the fluorescence may be measured by illuminating the GRH with light, e.g., excitation light having at least certain excitation wavelength spectrum, and measuring the fluorescence as a response to the illumination, e.g., the duration and/or intensity thereof. The excitation wavelength spectrum may depend on the chemical composition of the GRH. The excitation wavelength spectrum may lie in the visible range, e.g., as described in 10.1023/b:jofl.0000039338.16715.48, and/or in the NIR, e.g. as described in https://doi.org/10.1021/nn204323f.

For example, the GRH comprises boronic acid. For example, the GRH comprises boronic acid azobenzene. For example, the analyte-responsive hydrogel layer comprises boronic acid-substituted azobenzene derivatives. For example, the analyte-responsive hydrogel layer comprises o-boronic acid-substituted azobenzene. O-boronic acid-substituted azobenzenes are known to drastically change its absorption spectra in the visible range upon diol binding, as described in Egawa et al. “Colorimetric Sugar Sensing Using Boronic Acid-Substituted Azobenzenes”, Materials 2014, 7(2), 1201-1220; https://doi.org/10.3390/ma7021201.

The analyte-responsive layer 114 has a skin contacting surface 120 configured for directly or indirectly contacting the skin. For example, the skin contacting surface 120 may be the skin-contacting layer directly in contact with the skin of the user. Alternatively, additional layers between the skin contacting surface and the skin may be used, e.g., the skin contacting surface may be contacted indirectly via at least one additional element such as at least one additional light reflective layer 122.

The conversion layer 116 may be configured for converting the analyte. The conversion layer may actively remove the analyte from the analyte-responsive layer, thereby continuously re-creating an equilibrium analyte concentration. The analyte concentration may lie somewhere on the axis between the continuously varying analyte concentration on the leachate and “zero”, where the analyte is completely converted. The analyte-responsive layer 114 may be used for analyte detection, but the conversion layer 116 is required as an additional mechanism, which enable continuous equilibrium re-establishment.

The conversion layer 116 may be designed as a single stack of a functional layer, as shown in FIG. 1A. However, other embodiments are thinkable. For example, the conversion layer 116 may comprise two or more laterally distributed functional layers, as shown in FIG. 1B.

The conversion layer 116 may be enzyme-comprising or be non-enzymatic. For example, the conversion layer 116 may comprise enzyme molecules for converting of the analyte. The conversion layer 116 may comprise glucose oxidase (GOx) or glucose dehydroginase (GDH). In the case of the Gox, molecular oxygen from the ambient air can be used as natural electron acceptor. In this case, all layers above the conversion layer 116 may be designed to be at least partially oxygen permeably to supply oxygen to GOx. The reaction product, hydrogen peroxide, may, preferably, be directly decomposed in order to avoid its oxidative action towards enzyme. The conversion layer 116 may comprise some catalyst material, such as manganese oxide or platinum nanoparticles. In the case of GDH, which is an oxygen independent enzyme, another electron acceptor is desired, for instance silver chloride. In order to transfer electrons from the enzyme (co-factor) another mediator may be used. Therefore, the enzyme may be chemically wired by at least one redox hydrogel, e.g., at least one Os-complex modified hydrogel.

The conversion layer 116 may be non-enzymatic. For example, the conversion layer 116 comprises at least one material selected from the material classes of metal-organic frameworks, nanoparticle metal salts of low solubility, like tin-nickel sulfide, graphene oxide, carbon nanotubes and other.

The non-invasive analyte sensor 114 may comprise at least one electron acceptor 118. The conversion layer 116 may comprise an electron acceptor 118. The electron acceptor 118 may be a constituent part of the conversion layer 116. Depending on its design, the electron acceptor 118 may be made as a separate layer. The electron acceptor 118 may be configured to accept electrons generated by the conversion layer 116 during analyte, e.g., glucose, conversion. The electron acceptor 118 can be a constituent part of the analyte sensor 112, e.g., of the conversion layer 116 or an additional layer such as on top of the conversion layer 116. For example, the electron acceptor 118 is an Ag/AgCl layer. Additionally or alternatively, the electron acceptor may be an externally supplied commodity such as molecular oxygen.

The analyte sensor may comprise at least one light reflective layer 122. The light reflective layer 122 may be permeable for the analyte. For example, the light reflective layer 122 may be or may comprise at least one reflective membrane. The light reflective layer 122 may be arranged underneath of the analyte-responsive layer 114.

The non-invasive analyte sensing system 110 further comprises at least one optical measurement system 124 configured for detecting a change of the optical property of the analyte-responsive layer 114. Moreover, the non-invasive analyte sensing system 110 further comprises at least one processing unit 126 configured for determining an analyte concentration by evaluating the change of the optical property.

The optical measurement system 124 may comprise at least one light source. The light source specifically may be or may comprise at least one light-emitting diode (LED). Alternatively to an artificial light source, ambient light may be used. The light source may be configured for illuminating the analyte sensor 112. For example, light used for the typical purposes of the present disclosure is light in the visible range or the IR spectral range, e.g., the NIR range. A vast number of boronic acid based GRH composition may be possible, covering all possible wavelength ranges. The optical measurement system 124 may comprise at least one optical filter.

The optical property of the analyte-responsive layer 114 may be an optically measurable property which is determined as a function of wavelengths such as one or more of at least one property characterizing at least one reflectance property, at least one transmission property or at least one absorption property. The optical property may be determined for one or more wavelengths. For example, the optical property may be measurable by recording at least one absorption spectrum of the analyte-responsive layer 114. The optical measurement system 124 may comprise at least one spectrophotometer device. The spectrophotometer device may be an absorption spectrometer.

The optical measurement system 124 may comprise at least one camera. As an example, the camera may comprise at least one camera chip, such as at least one CCD chip and/or at least one CMOS chip. For example, the camera may be a color camera comprising at least three color pixels. The camera may be a color CMOS camera. The optical measurement system 124 may comprise at least one wavelength-selective element, such as at least one of a grating, a prism and a filter, e.g., a length variable filter having varying transmission properties over its lateral extension. The wavelength-selective element may be used for separating incident light into a spectrum of constituent wavelength signals whose respective intensities are determined by employing the camera.

The processing unit 126 may be configured for determining the physical property, e.g., the optical property such as a change of the optical property of the analyte-responsive layer 114. The determining of a change may comprise comparing the determined absorption spectrum to at least one reference spectrum. For example, the reference spectrum may be an absorption spectrum measured during manufacturing of the analyte sensor and/or at the begin of operation, e.g., right after mounting, of the analyte sensor on the skin. The reference spectrum may be stored in at least one database of the analyte sensing system and/or may be obtained from an external database such as from a cloud. For example, the reference spectrum may be a spectrum obtained by at least one further spectrometer, e.g., a reference spectrometer. The analyte sensing system may be calibrated using at least one further device, e.g., a blood glucose meter or another device for continuous glucose monitoring. In this case a spectrum obtained by the analyte sensing system may be correlated with a current analyte level measured by using the further device and make it thus to a reference.

The amount of change may be depend on the analyte concentration, e.g., may be proportional to the analyte concentration. For example, as outlined above, the GRH may change its fluorescence properties upon diol binding. The change of the fluorescence properties may be measured by illuminating the GRH with light, e.g., excitation light having at least certain excitation wavelength spectrum, and measuring the fluorescence as a response to the illumination. One or more properties of the fluorescence may be measured, e.g., a change of the intensity, change of the duration of the fluorescence, intensity decay over time and the like. Such properties may change as a function of the analyte concentration, e.g., as described in https://doi.org/10.1023/B:JOFL.0000039338.16715.48 FIG. 1.

Additionally or alternatively, at least one colorimetric measurement may be performed, e.g., a change in the absorption spectrum may be used. For example, in case of the, e.g., boronic Acid-Substituted Azobenzenes, a colorimetric measurement may be performed, i.e., a change in the absorption spectrum may be monitored. For example, some wavelength range of the absorption spectrum may be used or one or few single wavelengths of the absorption spectrum, e.g., absorption at 521 nm and/or 398 nm for a given compound.

The determined physical property may be evaluated into a concentration value by the processing unit, e.g., by using at least one predetermined relationship between the analyte concentration and the measured physical property.

FIG. 1A shows an embodiment in which the non-invasive analyte sensor 112 is a glucose responsive optical sensor. For example, the analyte sensor 112 comprises the following layers in the named order, starting most proximal from the skin to further distal layers. The analyte sensor 112 may comprise as most proximal layer to the skin the analyte-responsive layer 114, e.g., designed as an optical GRH layer. On top of the analyte-responsive layer 114 the conversion layer 116, e.g., a wired enzyme layer, may be arranged. On top of the conversion layer 116, the electron acceptor 118, e.g., an Ag/AgCl layer, may be used.

In the embodiment of FIG. 1A, the analyte-responsive layer 114 may be designed as an optical GRH layer changing its fluorescence properties, e.g., duration and/or intensity, upon diol binding. The optical measurement system 124 illuminates the GRH layer by an excitation wavelength spectrum and measures the intensity and foremost the duration and/or intensity thereof. The conversion layer 116, in this case an enzyme layer, converts the diffusing glucose. In addition, an Ag/AgCl layer is used which acts as an electron acceptor 118. Using GRH working in the IR range allows applying of Ag/AgCl on the top, as it is nearly transparent in the IR range.

For GRH working in visible spectral range, the Ag/AgCl layer may not lie in the pathway of the optical measurement system 124, as it will absorb the visible light. For example, as shown in FIG. 1B, the analyte sensor 112 comprises the following layers in the named order, starting most proximal from the skin to further distal layers. The analyte sensor 112 may comprise as most proximal layer to the skin the light reflective layer 122. On top of the light reflective layer 122, the analyte-responsive layer 114 may be arranged. On top of the analyte-responsive layer 114 the conversion layer 116, e.g., a wired enzyme layer, may be arranged. In the embodiment of FIG. 1B, e.g., for GRH working in visible spectral range, the Ag/AgCl layer may not lie in the pathway of the optical measurement system 124, as it will absorb the visible light. Therefore, the sensor 112 may comprise a transparent conductive layer 128, e.g., PEDOT or ITO/FTO coated transparent plastics or foils. This may allow placing the Ag/AgCl layer laterally shifted in respect to the GRH and light pathway. The optical measurement system 124 may operate in a reflective mode, sensing light with desirable wavelength towards the skin. The light passes through the transparent conductive substrate, the thin negligible absorbing enzyme layer and, finally, through the GRH. The passing light is being reflected at the reflective membrane and passes through the GRH, enzyme-comprising layer and the transparent window again and is being measured by the optical measurement system 124. By evaluation of the absorbance of the GRH, the analyte concentration can be evaluated. By the enzyme layer generated electrons are transported towards the Ag/AgCl layer through the conductive substrate.

FIG. 2 shows an embodiment of a method for determining a concentration of an analyte in a sample. The method comprises the method steps as listed as follows. The method steps may be performed in the given order. Further, one or more of the method steps may be performed in parallel and/or in a time overlapping fashion. Further, one or more of the method steps may be performed repeatedly. Further, additional method steps may be present which are not listed. The method comprising the steps of:
i. (130) contacting non-invasive analyte sensor 112 with a skin surface;
ii. (132) determining the physical property of the analyte-responsive layer 114;
iii. (134) determining the analyte concentration by evaluating the physical property.

### LIST OF REFERENCE NUMBERS

100 skin
110 non-invasive analyte sensing system
112 analyte sensor
114 analyte-responsive layer
116 conversion layer
118 electron acceptor
120 skin contacting surface
122 light reflective layer
124 optical measurement system
126 processing unit
128 transparent conductive layer
130 contacting
132 determining a change of the optical property
134 determining the analyte concentration

While this invention has been described as having an exemplary design, the present invention may be further modified within the spirit and scope of this disclosure. This application is therefore intended to cover any variations, uses, or adaptations of the invention using its general principles.

What is claimed is:

1. A non-invasive analyte sensor for on-skin wearing comprising:
at least one analyte-responsive layer exhibiting at least one physical property dependent on a quantity of the analyte, wherein the analyte-responsive layer has a skin contacting surface configured for directly or indirectly contacting the skin;
at least one conversion layer; and
wherein the analyte responsive layer is an equilibrium analyte-responsive layer, wherein the analyte-responsive layer comprises at least one analyte-responsive hydrogel layer, wherein the analyte-responsive hydrogel layer comprises boronic acid, wherein the analyte-responsive hydrogel layer comprises a phenylborate derivative.

2. The non-invasive analyte sensor according to claim 1, wherein the conversion layer is enzyme-comprising.

3. The non-invasive analyte sensor according to claim 1, wherein the conversion layer is non-enzymatic.

4. The non-invasive analyte sensor according to claim 1, wherein the conversion layer comprises enzyme molecules for converting of the analyte, wherein the conversion layer comprises glucose oxidase (GOx) or glucose dehydroginase (GDH).

5. The non-invasive analyte sensor according to claim 4, wherein the enzyme is chemically wired by at least one redox hydrogel.

6. The non-invasive analyte sensor according to claim 1, wherein the non-invasive analyte sensor comprises at least one electron acceptor, wherein the electron acceptor is a redox active chemical compound configured for accept electrons generated by the conversion layer.

7. The non-invasive analyte sensor according to claim 6, wherein the conversion layer comprises the electron acceptor.

8. The non-invasive analyte sensor according to claim 7, wherein the electron acceptor is an Ag/AgCl layer.

9. The non-invasive analyte sensor according to claim 6, wherein the electron acceptor is an Ag/AgCl layer.

10. The non-invasive analyte sensor according to claim 1, wherein the analyte sensor comprises at least one light reflective layer, wherein the light reflective layer is permeable for the analyte.

11. The non-invasive analyte sensor according to claim 1, wherein the analyte is glucose.

12. The non-invasive analyte sensor according to claim 1, wherein the sample is a bodily leachate.

13. The non-invasive analyte sensor according to claim 12 wherein the bodily leachate is sweat.

14. A non-invasive analyte sensing system comprising:
at least one non-invasive analyte sensor according to claim 1,
at least one optical measurement system configured for detecting the physical property of the analyte-responsive layer;
at least one processing unit configured for determining an analyte concentration by evaluating the physical property.

15. A method for determining a concentration of an analyte in a sample using a non-invasive analyte sensing system according to claim 14, the method comprising the steps of:
i. contacting non-invasive analyte sensor with a skin surface;
ii. determining the physical property of the analyte-responsive layer;
iii. determining the analyte concentration by evaluating the physical property.

16. A non-transitory computer readable medium having stored thereon computer executable instructions for performing steps ii. and iii. of the method according to claim 15.
